SEQUENCE LISTING (1 sequence)

SEQ ID NO: 1 - protein, 689 aa, Homo sapiens
MAPWPELGDAQPNPDKYLEGAAGQQPTAPDKSKETNKNNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCGCPKCCRCSKCCEDLEEAQEGQDVPVKAPETFDNITISREAQGEVPASDSKTECTAL

(19) United States
(12) Patent Application Publication
(10) Pub. No.: US 2024/0103015 A1
(43) Pub. Date: Mar. 28, 2024

(54) METHODS FOR ASSAYING SLC34A2 EXPRESSION IN MUSCLE TISSUE AND TREATING FACIOSCAPULOHUMERAL MUSCULAR DYSTROPHY

(71) Applicant: University of Maryland, Baltimore, Baltimore, MD (US)

(72) Inventors: Robert J. Bloch; Andrea O'Neill, New Freedom, PA (US); Maria Traficante, Towson, MD (US)

(21) Appl. No.: 18/477,206

(22) Filed: Sep. 28, 2023

Related U.S. Application Data
(60) Provisional application No. 63/410,787, filed on Sep. 28, 2022.

(51) Int. Cl. G01N 33/68; A61K 49/00; C07K 16/28

(52) U.S. Cl. CPC G01N 33/6887; A61K 49/0002; C07K 16/28; G01N 2800/2878

(57) ABSTRACT

The present invention is directed to methods of detecting SLC34A2 expression in muscle tissue in a sample from a subject comprising providing the sample from the subject; and conducting an assay to detect an expression level of SLC34A2 in the sample.

### CROSS-REFERENCE TO RELATED APPLICATIONS

This application claims the benefit of U.S. Provisional Application No. 63/410,787, filed Sep. 28, 2022, the contents of which are hereby incorporated by reference in their entirety.

### INCORPORATION-BY-REFERENCE OF MATERIAL SUBMITTED ELECTRONICALLY

Incorporated by reference in its entirety herein is a computer-readable sequence listing and identified as follows: One 2,686 Byte XML file named “Sequence_listing.xml,” created on Sep. 25, 2023.

### FIELD OF THE INVENTION

This field of the invention relates to medicine and more specifically to the disease of facioscapulohumeral muscular dystrophy (FSHD) and for diagnostic methods, therapeutics and methods of treating the same.

### BACKGROUND OF THE INVENTION

Muscular dystrophies (MDs) are a group of genetic diseases. The group is characterized by progressive weakness and degeneration of the skeletal muscles that control movement and breathing. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Muscular dystrophies afflict approximately 1 in 5000 individuals worldwide. The development of models, diagnostic assays, and markers to study and diagnose these diseases is essential in understanding the mechanisms of pathogenesis and to test potential therapies for people afflicted with these diseases. Animal models for some muscular dystrophies, such as facioscapulohumeral muscular dystrophy (“FSHD”) and oculopharyngeal muscular dystrophy (“OPMD”), are limited. Furthermore, some murine models of some muscular dystrophies, whether naturally occurring or genetically engineered, are limited because they do not replicate all the features of the human disease.

Facioscapulohumeral Muscular Dystrophy (FSHD) is the third most common muscular dystrophy in the human population, with about 1 in 8300 people affected. It usually does not affect life span but the disability it engenders seriously affects quality of life. Determining its natural history, and then developing treatments to slow, stop and reverse its course, are major goals.

Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

A key feature of FSHD is that it is epigenetic in origin. Contractions at the end of human chromosome 4q lead to demethylation of 3300 bp, D4Z4 repeats, which encode DUX4, a transcription factor that is normally only expressed during the four-cell stage of human development but repressed thereafter FSHD is thus caused by aberrant expression of DUX4. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

Despite progress in the FSHD field, there are still no approved treatments for FSHD, and therapeutic development remains a critical need in the field. What is needed are new models, methods for diagnosis and monitoring progression of FSHD, and assays for screening potential therapeutics to treat FSHD. The foregoing description of the background is provided to aid in understanding the invention and is not admitted to be or to describe prior art to the invention.

### SUMMARY OF THE INVENTION

It is to be understood that both the foregoing general description of the invention and the following detailed description are exemplary, and thus do not restrict the scope of the invention.

Facioscapulohumeral Muscular Dystrophy (FSHD) is caused by the derepression of the DUX4 gene in one or a few muscle fibers pre muscle at any given time. Its sporadic expression initiates the cascade of genes, eventually leading to fiber death. Understanding and monitoring of these early stages of pathogenesis would greatly facilitate the development of therapies for FSHD.

One of the genes upregulated by DUX4 is SLC34A2, a sodium-dependent phosphate transporter that in adults is normally only expressed in epithelia and in some cancers. It is shown herein that SLC34A2 is expressed 10 times more in FSHD fibers than healthy fibers, in both biopsies and xenografts generated in mice. The protein appears in two distinct patterns in immunoflourescently labeled cross sections of FSHD fibers: (a) on the surface of myofibers and in a clear reticulum across the myoplasm, identical to transverse tubules in healthy fibers; (b) in an intensely labeled aggregate that fills the myoplasm. The latter pattern appears in fibers that are clearly necrotic. Without being bound by theory, it is believed the former represents fibers in which the DUX4 program has been activated but has not yet led to fiber death.

SLC34A2 is a multi-pass transmembrane protein with an extracellular epitope that in epithelia and cancers is bound by a monoclonal antibody (mAb), MX35. MX35 binds to intact cells and is FDA approved. MX35 in its humanized form, Rebmab200, has been used to track metastasis and for ovarian cancer immunotherapy. If it is radioactively labeled, it can be detectable by PET or SPECT imaging.

In one aspect, the invention provides a method for monitoring FSHD using SLC34A2 as a biomarker for the disease.

In another aspect, the invention provides a method of detecting SLC34A2 expression in living muscle tissue in a subject comprising
i) administering to the subject an effective amount of an agent that binds to SLC34A2 in the muscle tissue; and
ii) detecting the agent in the muscle tissue in situ, thereby detecting SLC34A2 expression in living muscle tissue.

In another aspect, the invention provides a method of detecting SLC34A2 expression from muscle tissue in blood, plasma or serum from a subject comprising
i) providing a blood, plasma or serum sample from the subject; and
ii) conducting an assay to detect a level of SLC34A2 in the blood, plasma or serum sample.

In another aspect, the invention provides a method of detecting SLC34A2 expression in muscle tissue in a sample from a subject comprising
i) providing the sample from the subject; and
ii) conducting an assay to detect an expression level of SLC34A2 in the sample.

In another aspect, the invention provides a method for monitoring the presence of FSHD in myofibers at levels of expression found at higher levels than in controls. Specifically, the method utilizes antibodies, such as fluorescently tagged MX35, which can specifically bind to cells expressing SLC34A2.

In certain embodiments of the instant invention, a fluorescently-tagged MX35 can be used to label myofibers in mice that express DUX4 transgenically, following viral transduction, or that have been xenografted to develop into mature human FSHD myofibers. Use of different fluorescent tags followed by in vivo imaging with IVIS instrumentation will allow for the monitoring and appearance of fibers expressing SLC34A2 as a biomarker for FSHD, and for following the progression of SLC34A2 expression in individual muscles and from muscle to muscle. Additionally, fluorescently-labeled MX35 or Rebmab200 can be used to determine the efficacy of treating mice with different p38 kinase inhibitors such as losmapimod which reduces the DUX4 program in vitro and is now in clinical trial.

In another aspect, the invention relates to other proteins which can be used as biomarkers for FSHD, these include but are not limited to: PRAMEF-12, IL-6, HSP70, MMP2, CK, Trpn2, PKCa, PKCp11, BTRK, and SRC.

In another aspect, the invention is a method for monitoring the progression or severity of FSHD in a subject, such as a mouse or human, using the biomarker SLC34A2 and molecules which can bind to SLC34A2 and be visualized in immunofluorescence-type assays. These assays can be formulated to work with other biomarkers for FSHD such as PRAMEF-12, IL-6, HSP70, MMP2, CK, Trpn2, PKCa, PKC11, BTRK, and SRC.

In another aspect, the invention is a method for identifying molecular modulators of the DUX4 pathogenic program by measuring the expression of the biomarker SLC34A2, which is reduced in expression when the DUX4 pathogenic program is inhibited by an inhibitor such as a p38 kinase inhibitor.

In another aspect, the invention is a method for monitoring the expression of the biomarker SLC34A2 for FSHD by measuring the amount of the biomarker in a frozen section of a xenograft in a mouse model of FSHD using a monoclonal antibody such as MX35 or Rebmab200 which binds to SLC34A2.

In another aspect, the invention is a method to mark FSHD tissues in situ using a fluorescently tagged MX35 monoclonal antibody which binds the SLC34A2 protein.

In another aspect, the invention is a method for using the fluorescently tagged MX35 or Rebmab200 antibody for tracking the appearance and disappearance of positive fibers for FSHD over time and for assessing the effectiveness of therapies which can suppress the DUX4 program that is responsible for the expression of SLC34A2.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Shown in panels (A) and (B) show SLC34A2 protein quantitated in frozen sections of xenografts of FSHD (15A, C6) and control (15V, A4) cells. Arrows in B point to labeled fibers. The results in (C) and (D) show that FSHD xenografts have about 10-times more fibers that label for SLC34A2 than control xenografts * p<0.05.

FIG. 2. Immunoblotting of SLC34A2 in human serum. The results show that human sera from individuals with FSHD tend to have more SLC34A2 protein, normalized to the amount of human serum albumin, than sera from healthy individuals.

FIG. 3. Quantitation of SLC34A2 in human serum from control and FSHD patients, from blots as in FIG. 2. Blotting was with the antibody to the C-terminus of SLC34A2. The results show a significant difference between FSHD and healthy controls (p=0.0065).

FIG. 4. LY222820 (ralimetinib) reduces expression of the mRNAs encoding DUX4 target genes, including SLC34A2, in FSHD xenografts, as assayed by qPCR. *, p<0.05; ** p<0.01.

FIG. 5. Decreased expression of the SLC34A2 protein in cultured C6 (FSHD) myotubes treated with three inhibitors of p38 kinase: losmapimod, pamapimod, and ralimetinib . ****, p<0.001.

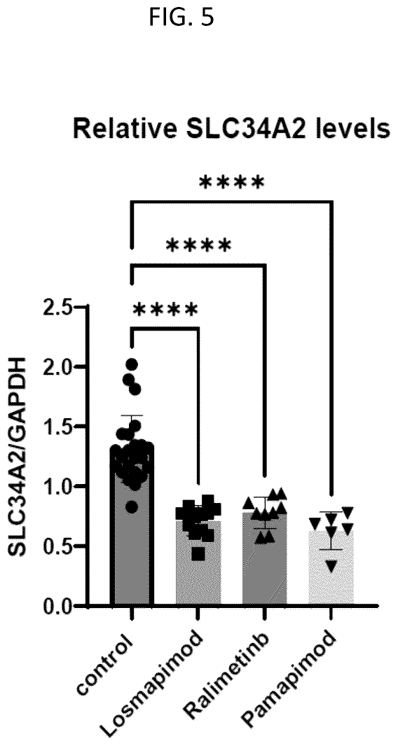

FIG. 6. Frozen sections of FSHD xenografts labeled with MX35, a human anti-SLC34A2 antibody that recognizes an extracellular epitope of SLC34A2 (green), counterstained with rabbit antibody to its C- terminal sequence (red). The result show examples of a myofiber labeled at its surface and in an internal reticulum (largest fibers in A,A′, B, B′) as well as other fibers with condensed labeling typical of necrotic fibers.

FIG. 7. Engrafted muscle, as in FIG. 6, was first labeled by IM injection of MX35-647, a fluorescent form of MX35 prepared by conjugation with IR-647, into the engrafted limb, then collected, sectioned and immunolabeled with anti-human β-spectrin and anti-human Lamin A/C, to identify the FSHD fibers in the graft (red), and antibody to the C-terminal sequence of SLC34A2 (green). MX35-647 is shown in purple. DAPI labeling of nuclei is in blue.

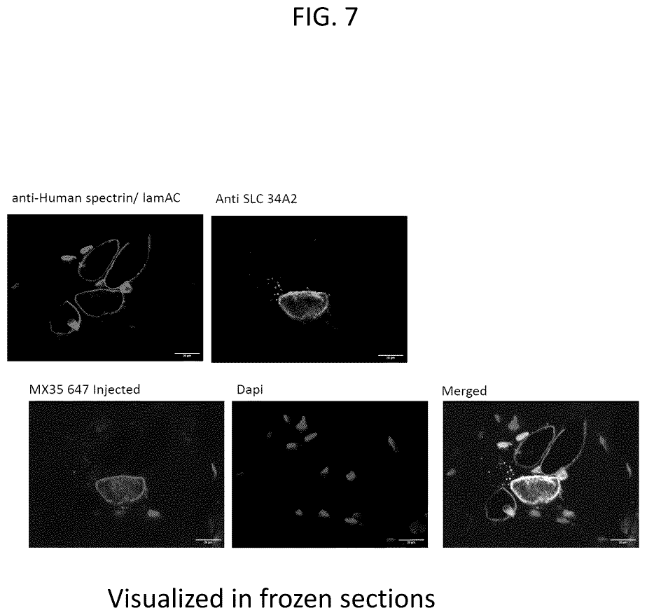

FIG. 8. IVIS visualization of SLC34A2 in the muscles of living mice with MX35-647, 2 days after injection of Ab. The right TA was injected with antibody. The left hindlimb, which also carried an FSHD xenograft, was not. The results show that the antibody labels the engrafted limb in which it was injected but it does not spread significantly to the uninjected limb.

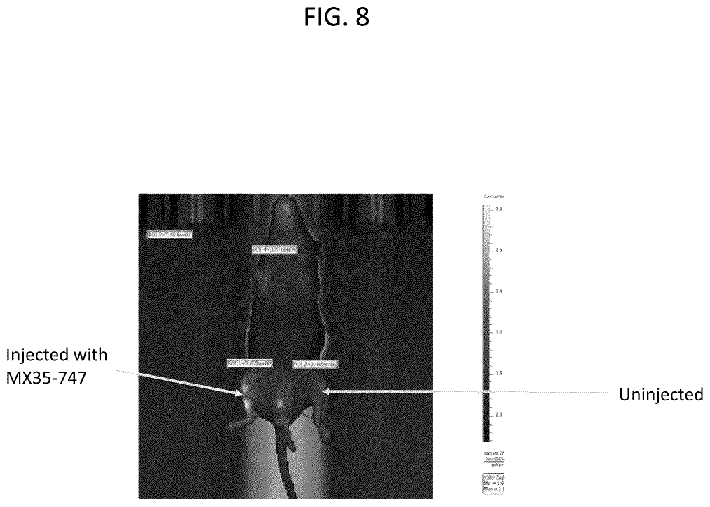

FIG. 9. SLC34A2 protein in grafts and serum, assayed by immunoblotting with antibody to the C-terminus. The results show that the protein is detected in xenografts and at higher levels in the serum of a mouse carrying FSHD xenografts than in the serum of a mouse carrying control xenografts.

FIG. 10. Linearity of immunoblot assay of SLC34A2 in serum. The results show that the assay of both SLC34A2 and human serum albumin are linear over the range we used in our assays shown in FIGS. 2 and 3

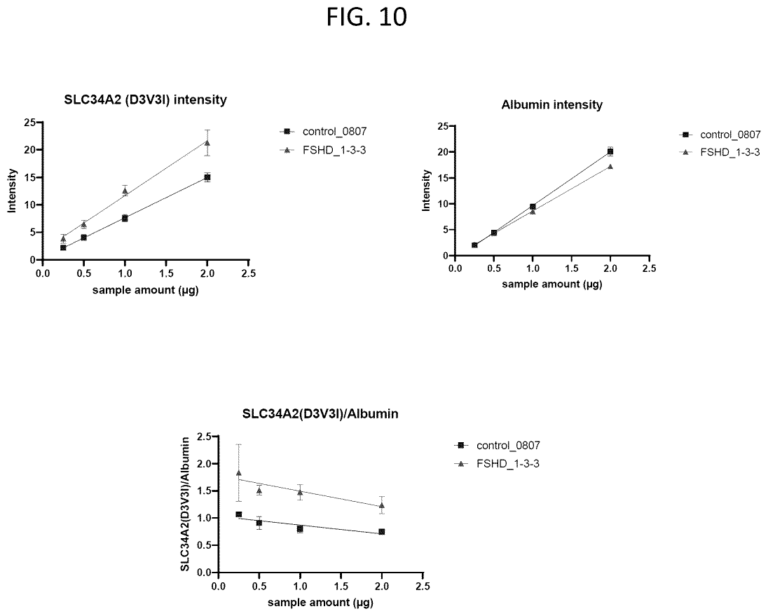

FIG. 11. The Muscle Xenograft Model: Protocol. Approach: Create a niche to inject immortalized human muscle precursor cells from FSHD patients, into the mouse hindlimb, and allow them to grow, fuse, and form mature human muscle xenografts.

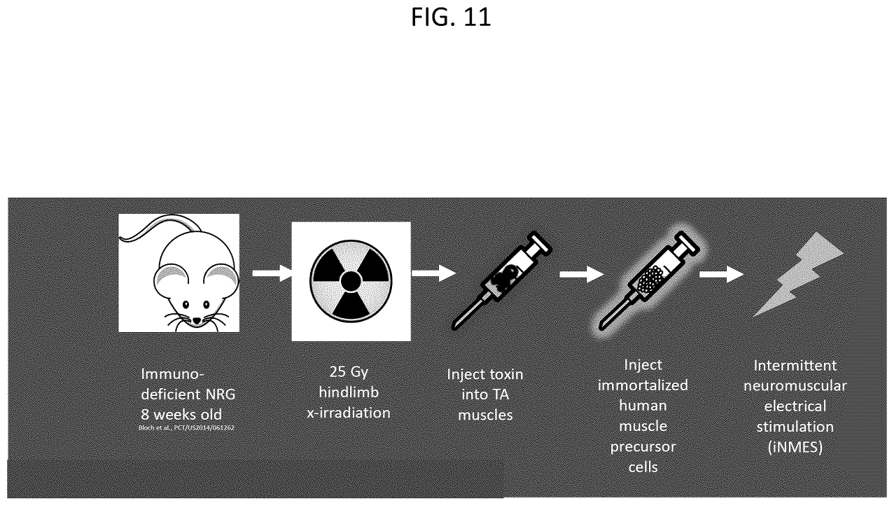

FIG. 12. FSHD (top) and control (bottom) myotubes in culture labeled with mAb MX35 and rabbit antibodies to the N-terminus of SLC34A2 after fixation and permeabilization. Control myotubes are visualized with fluorescent wheat germ agglutinin (WGA). Bars, 100 μm. The data show that FSHD but not control, healthy myotubes express SLC34A2 protein, as assayed by immunofluorescence labeling with antibodies to the N-terminus of SLC34A2, and with the MX35 mAb, to an extracellular epitope of the protein

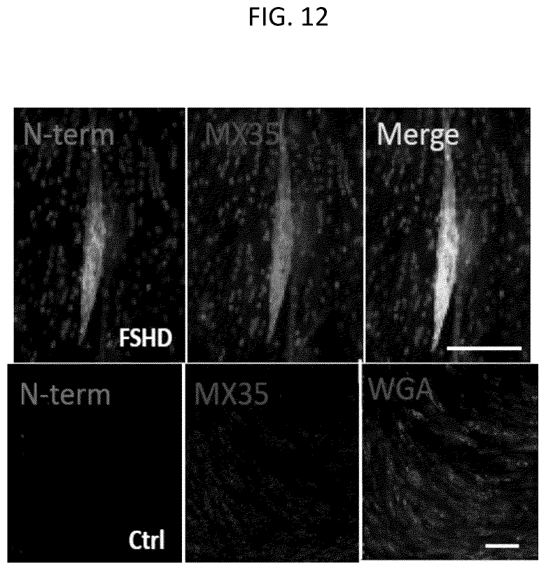

FIG. 13. Immunoblots of FSHD and control myotubes with antibodies to the C- and N-termini of SLC34A2 (A) and after immunoprecipitation with MX35 (B), followed by blotting. The data show that the N- and C-terminal specific antibodies recognize SLC34A2 specifically in immunoblots, and that the MX35 Ab can immunoprecipitated SLC34A2 from extracts of FSHD muscle cells in culture.

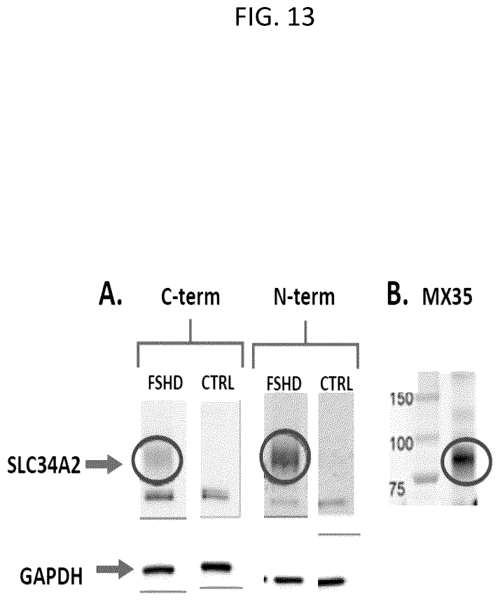

FIG. 14. Immunolabeling of living FSHD (C6) and control (A4) myotubes with MX35 and control IgG. DAPI was used as a counterstain (blue). Bound MX35 and control IgG were recognized with Alexa-568 labeled goat anti-mouse IgG (red). The data shows that MX35 specifically labels the surface of intact living FSHD myotubes, but not control, healthy myotubes.

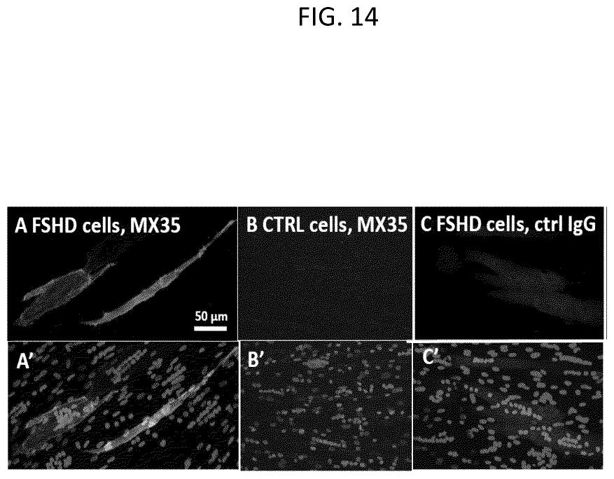

FIG. 15. Fibers labeled with MX35-647 in FSHD xenografts in living mice, in situ. (A-E): Fiber with labeling of SLC34A2 at sarcolemma and in the spotty/reticular pattern of t-tubules (F): Two fibers, similarly labeled, one necrotic (white arrow), the other not (yellow arrow). Scale:1 cm=50 μm. The results show that a systemic injection of a fluorescently tagged version of MX35 (MX35-650) labels myofibers in FSHD grafts in situ. Spectrin labeling in panel A identifies the fibers in the engrafted region that are human in origin. The fiber labeled with MX35-650 also labels with the antibody to the C-terminus of SLC34A2. This indicates that MX35 accesses the surfaces of living myofibers in FSHD-engrafted limbs.

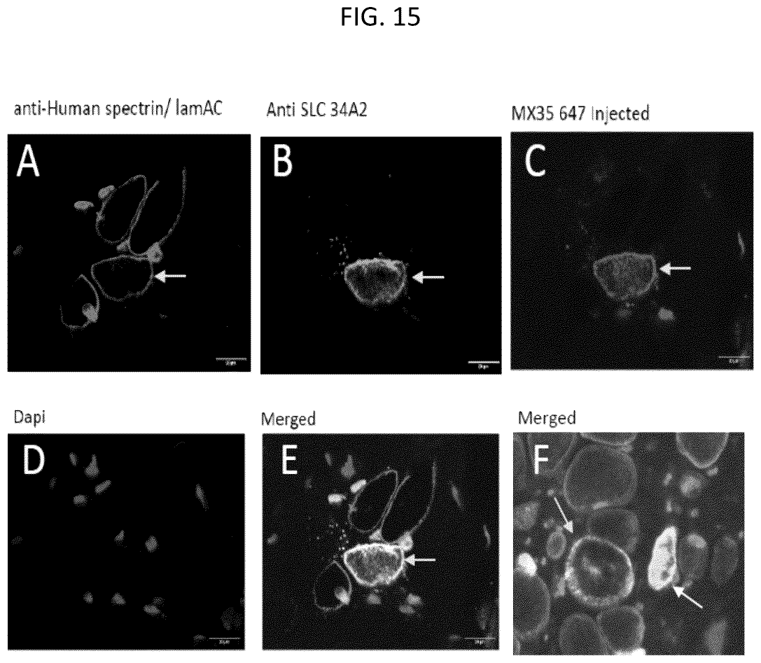

FIG. 16. Mouse carrying a healthy control graft in the right TA (Ctrl) and an FSHD graft in the left TA, IVIS-imaged 1 hr after injection with MX35-647. The results show that the FSHD leg is more intensely labeled, consistent with the greater ability of MX35-647 to recognize SLC34A2 in FSHD tissue.

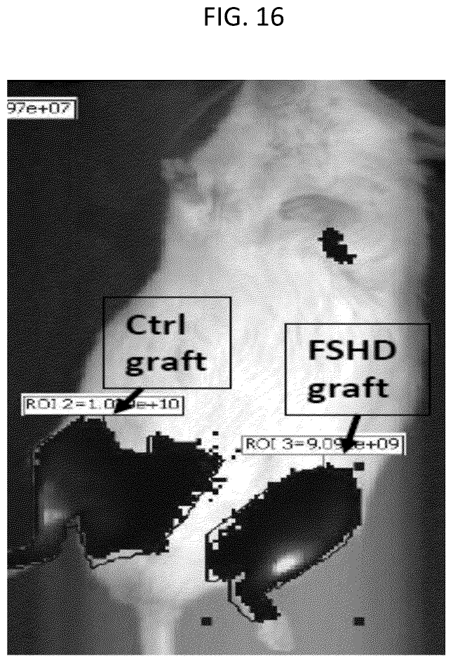

FIG. 17. Time course of antibody fluorescence labeling of FSHD xenografts in mice injected with fluorescent control IgG or MX35, measured by IVIS. Red, control IgG-647; black, MX35-647. The results show the fluorescence intensities measured from a single mouse over time. The results show that MX35-647 is retained at higher levels over longer periods of time in the FSHD graft than the control IgG-647.

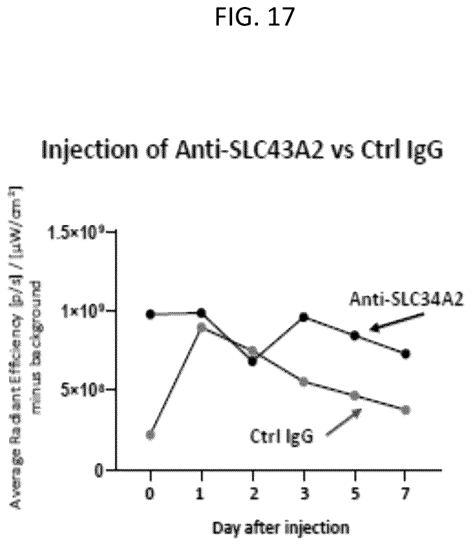

FIG. 18. As in FIG. 17, but repeated with 3 mice per group. FSHD grafts in both hindlimbs labeled poorly with the nonimmune control IgG-647 , compared to MX35-647, by IVIS imaging. The data show two sets of 3 mice carrying FSHD grafts in each leg, injected with either control IgG-650 or MX35-650. The intensities of the latter are generally higher, consistent with the ability of the MX435-650 to bind to some of the FSHD myofibers in the FSHD grafts.

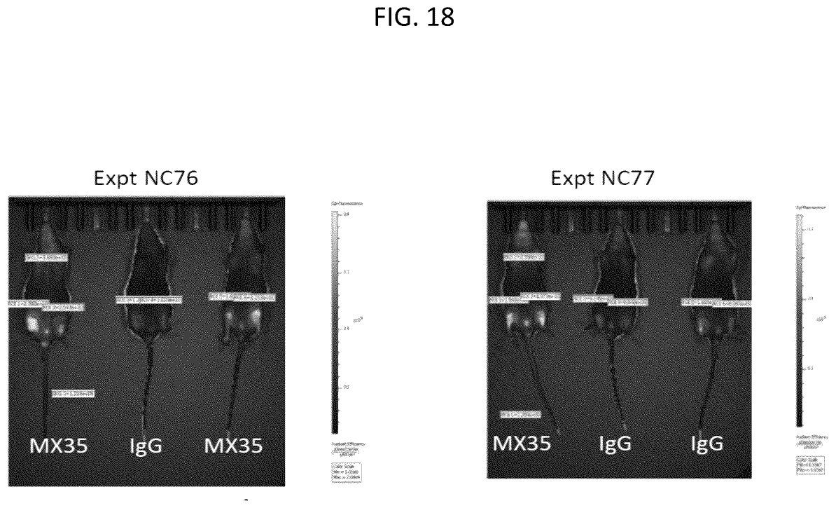

FIG. 19. A. Time course of antibody fluorescence in mice shown in FIG. 18, measured by IVIS. B. Quantitation of areas under each curve shown in A. The difference between the control IgG and MC35 is highly significant (p=0.002).

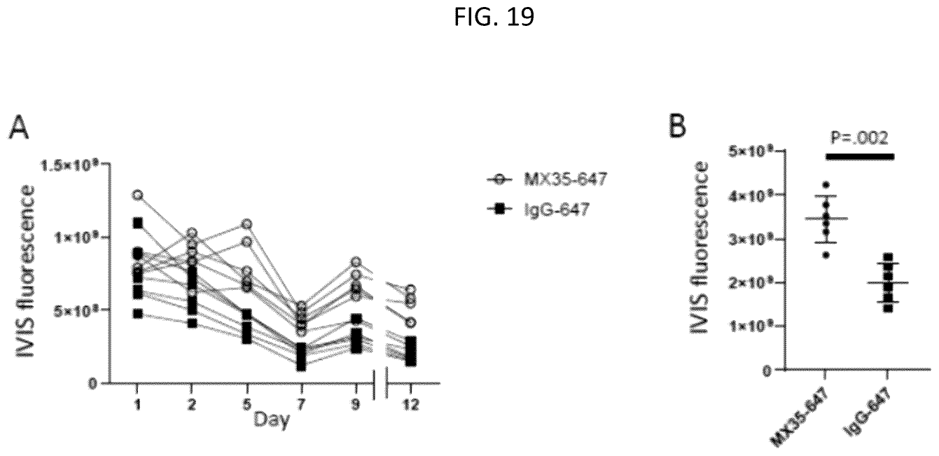

FIG. 20. Labeling of SLC34A2 protein at the surface of FSHD myotubes, with and without treatment with losmapimod. The results show robust labeling of one myotube in the FSHD sample, consistent with its expression in only a small number of muscle cells that activate that DUX4 program. Losmapimod reduces this signal significantly.

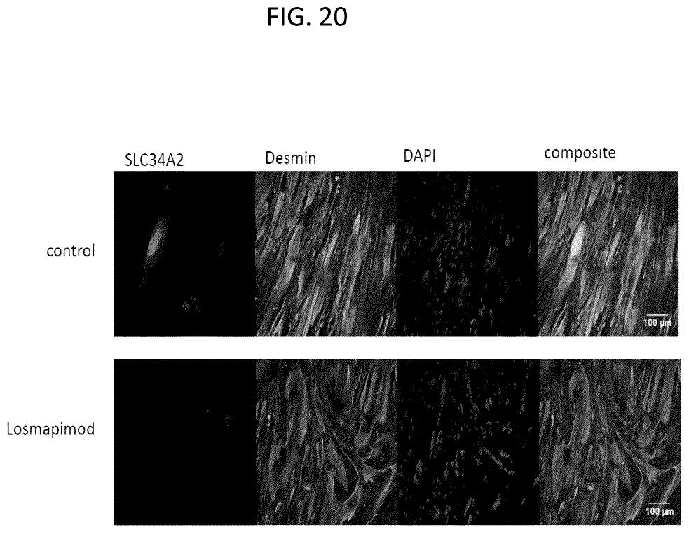

FIG. 21. Relative intensities of surface labeling of SLC34A2 in FSHD myotubes with and without losmapimod treatment, as in FIG. 20, normalized to the desmin signal in each ROI. *, p<0.05.

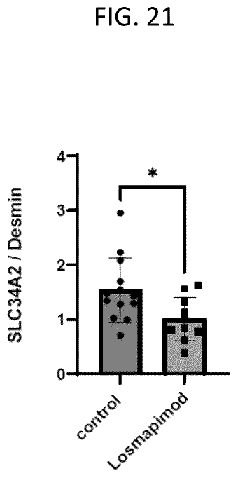

FIG. 22. Relative mRNA expression of DUX4, SLC34A2, and other downstream gene products of DUX4, in the presence and absence of losmapimod. *, p<0.05, **, p<0.01. The data show that losmapimod treatment of FSHD myotubes reduces the mRNAs encoding DUX4 and SLC34A2, as well as several other gene products downstream of DUX4.

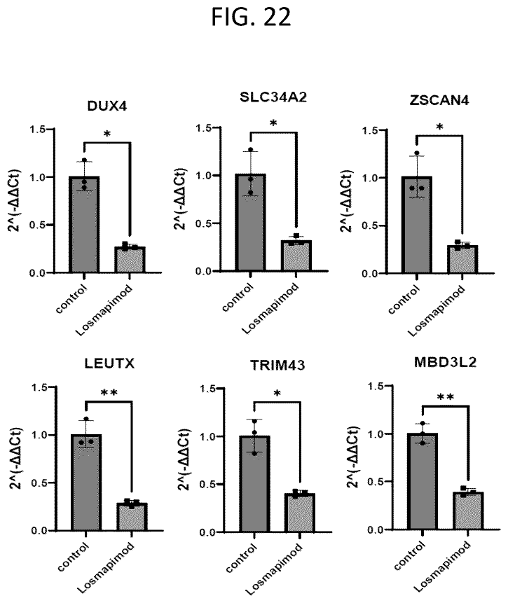

### DETAILED DESCRIPTION OF THE INVENTION

The present disclosure is based on the discovery that the protein SLC34A2 is a reliable biomarker for facioscapulohumeral muscular dystrophy (FSHD), caused by upregulation of the DUX4 gene, and further that SLC34A2 expression can be assayed in situ or in plasma, blood or serum to monitor progression of FSHD as well as screen for drugs to treat FSHD. The ability to observe FSHD in diseased muscle tissue in living animals has substantial utility for determining for determining which muscles in FSHD patients are actively experiencing pathology, the progression of the disease, and for determining which potential therapeutics can provide the greatest benefit for treatment. It may also be useful in determining the rate at which diseased muscle tissue can be replaced by healthy muscle tissue over time, once methods or replacement become available. In addition, the present disclosure encompasses methods that provide a quantitative measure of SLC34A2 in different biological samples such as blood, plasma or serum.

Reference will now be made in detail to embodiments of the invention which, together with the drawings and the following examples, serve to explain the principles of the invention. These embodiments describe the invention in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art. For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. Al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

In one embodiment, the invention provides a method for monitoring FSHD using SLC34A2 as a biomarker for the disease.

In another embodiment, the invention provides a method of detecting SLC34A2 expression in living muscle tissue in a subject comprising
i) administering to the subject an effective amount of an agent that binds to SLC34A2 in the muscle tissue; and
ii) detecting the agent in the muscle tissue in situ, thereby detecting SLC34A2 expression in living muscle tissue.

In another embodiment, the invention provides a method of detecting SLC34A2 expression from muscle tissue in blood, plasma or serum from a subject comprising
i) providing a blood, plasma or serum sample from the subject; and
ii) conducting an assay to detect a level of SLC34A2 in the blood, plasma or serum sample.

In another embodiment, the invention provides a method of detecting SLC34A2 expression in muscle tissue in a sample from a subject comprising
i) providing the sample from the subject; and
ii) conducting an assay to detect an expression level of SLC34A2 in the sample.

As used herein, the terms “animal,” “patient,” or “subject,” mean any animal (e.g., mammals, including but not limited to humans, primates, dogs, cattle, cows, horses, kangaroos, pigs, sheep, goats, cats, rabbits, rodents, and transgenic non-human animals). Typically, the terms “animal,” “subject,” and “patient” are used interchangeably herein in reference to a human subject or other animal. The subject is not particularly limiting. In some embodiments, the subject is a mouse or other experimental animal, such as, for example, a rat, pig minipig, or monkey. In some embodiments, the subject is a mammal, such as a human.

As used herein, the term “sample” refers to any type of material of biological origin isolated from a subject, including, for example, DNA, RNA, protein, such as, for example, blood, plasma, serum, fecal matter, urine, semen, bone marrow, bile, spinal fluid, tears, saliva, muscle biopsy, organ tissue or other material of biological origin known by those of ordinary skill in the art.

In some embodiments, the subject has a muscle disorder or disease. As used herein, the term “muscle disorder” is intended to broadly encompass muscle disease, muscle injuries, and defects that can impair and reduce muscle function including but not limited to physical injuries, burns, surgical tissue excisions, muscle wasting, muscular dystrophy, infarcts, ischemic events, neuromuscular disorders and muscle diseases.

In some embodiments, the subject is a human and is diagnosed as having Facioscapulohumeral Muscular Dystrophy (FSHD). Without being bound by theory, it is believed that FSHD is caused by the derepression of the DUX4 gene in one or a few muscle fibers at any given time. Its sporadic expression initiates a cascade of genes, eventually leading to fiber death.

In some embodiments, the subject is an animal model. In some embodiments, the subject is a xenografted animal.

As used herein, the term “xenograft” or “xenotransplant” refers to a transplanted cell, tissue, or organ derived from an animal of a different species. By way of an example, a graft from a mouse to a human is a xenograft. As used herein, the term “xenotransplantation” refers to the process of transplantation of living cells, tissues or organs from one species to another, such as from mice to humans.

As used herein, the term “engrafting” or “engraftment” is used herein to refer to the ability of cells or tissue, such as hMPCs or LHCN cells, provided by transplantation, to repopulate a tissue. The term encompasses all events surrounding or leading up to engraftment, such as tissue homing of cells, colonization of cells within the tissue of interest, and growth and differentiation of these cells into mature tissue. The engraftment efficiency or rate of engraftment can be evaluated or quantified using any experimentally acceptable parameter as known to those of skill in the art and can include cellular number and size. If the engrafted cells are engineered to express biologically active compounds, engraftment can also be quantified by the effects of these compounds, e.g. , on the survival of the recipient. In one embodiment, engraftment is determined by measuring bioluminescence during a post-transplant period.

As used herein, the term “disease model” refers to the use of non-human animal models to obtain new information about human muscular diseases.. In some embodiments, a population of cells from dystrophic patients, generated and immortalized as the LHCN cells were, and injected into mice following the methods as disclosed herein, can be used in disease modeling experiments.

In some embodiments, the muscle tissue is human muscle tissue that has been engrafted into an animal, such as a mouse. In some embodiments the muscle tissue is derived from precursor cells that have been engrafted. In some embodiments, the muscle tissue is made from human muscle precursor cells (hMPC), e.g., from a subject having facioscapulohumeral muscular dystrophy (FSHD). U.S. Pat. No. 10,632,305 describes methods to promote the engraftment and development of myogenic cells from individuals with muscular dystrophies into mature muscle tissue in mice to study and treat muscle diseases, muscle injury and reduced muscle function, which disclosure is incorporated by reference in its entirety.

The present disclosure provides that one of the genes upregulated by DUX4 is SLC34A2, which is a Na+, Pi-cotransporter that in adults is normally only expressed in epithelia and in some cancers. SLC34A2 is expressed 10 times more in FSHD fibers than healthy fibers, in both biopsies and xenografts generated in mice. The protein appears in two distinct patterns in immunoflourescently labeled cross sections of FSHD fibers: (a) at the surface of the myofiber and in a clear reticulum across the myoplasm, identical to transverse tubules in healthy fibers; (b) in an intensely labeled aggregate that fills the myoplasm. The latter pattern appears in fibers that are clearly necrotic. Without being bound by theory, it is believed the former represents fibers in which the DUX4 program has been activated but has not yet led to fiber death.

SLC34A2 is a multi-pass transmembrane protein with an extracellular epitope. This polypeptide is also known as NAPI-3B, NAPI-Ith, NPTIIb, NaPi2b, and PULAM. In some embodiments, SLC34A2 has the amino acid sequence set forth in NCBI Accession No.: BC146666.1 (Gene ID: 10568) (SEQ ID NO:1).

As used herein, “muscle cells” or “muscle tissue” includes, but are not limited to, skeletal muscle fibers, myofibers, myocytes, myoblasts or myotubes, and may be of any suitable species, and in some embodiments are of the same species as the subject into which tissues are implanted. Mammalian cells (including mouse, rat, pig or minipig, dog, cat, monkey and human cells) are in some embodiments particularly preferred.

The term “tissue” refers to a group or layer of similarly specialized cells which together perform certain special functions. The term “tissue-specific” refers to a source or defining characteristic of cells from a specific tissue.

As used herein, the term “muscle fiber” or “myofiber” refers to a multinucleated single muscle cell. Physically, e.g., in humans, they are highly elongated and are typically 50-100 microns in diameter, but range in length from a few millimeters many centimeters. Muscle fiber cells are formed from the fusion of myoblasts (a type of progenitor cell that gives rise to a muscle cell during development or, in adults, during regeneration following injury). The myofibers are long, cylindrical, multinucleated cells composed largely of actin and myosin myofibrils organized and repeated in many sarcomeres, the basic functional unit of the muscle fiber and responsible for skeletal muscle's striated appearance and forming the basic machinery necessary for muscle contraction.

As used herein, the term “myoblasts” are a type of muscle precursor cell. They are present in developing muscle and appear in adult muscle when satellite cells, muscle stem cells that are closely associated with myofibers in vertebrates, become activated. If the myofiber is injured, the myoblasts are capable of dividing and fusing to form a new myofiber. Typically, after muscle injuries, myofibers become necrotic and are removed by macrophages. This induces activation of stem cells and the proliferation and fusion of myoblasts to form multinucleated and elongated myotubes, which develop further into myofibers. The myofibers so generated form a more organized structure, namely muscle.

As used herein, the term “myocytes” are muscle cells, muscle fibers, or skeletal muscle cells, in either the mature or immature state.

As used herein, the term, “myofibrils” are the slender threads of a muscle fiber composed of numerous myofilaments. Myofibrils run from one end of the cell to the other and attach to the cell surface membrane at each end.

As used herein, the term “myotubes” are elongated, multinucleated cells, normally formed by the fusion of myoblasts. Myotubes generally have centrally located nuclei and myofibrils that tend to be poorly organized. In vertebrates, they develop into mature muscle fibers, which have peripherally-located nuclei and myofibrils that are well organized (e.g. , in mammals). Under low serum conditions, myoblasts exit the cells cycle and fuse to form multinucleated myotubes, which become contractile.

In some embodiments, the level of SLC34A2 can be assayed using techniques to detect proteins or nucleic acids, such as, for example, PCR, northern blotting, in situ hybridization, immune-based detection, fluorescence detection, in vivo imaging, scanning, and the like. In some embodiments, SLC34A2 expression is assayed using an agent that binds to SLC34A2, such as an antibody.

In some embodiments, SLC34A2 expression is detected in situ in living muscle tissue using a binding agent, such as, for example, a labeled antibody. In some embodiments, SLC34A2 expression is analyzed using the IVIS® Spectrum in vivo imaging system (Perkin Elmer), magnetic resonance, computed tomography, or positron emission tomography scanning.

In some embodiments, the assay comprises contacting the blood, plasma or serum sample with an agent that binds to SLC34A2, and detecting the binding, or administering to the subject an agent that binds to SLC34A2 in situ in living muscle tissue.

In some embodiments, SLC34A2 expression in blood, plasma, serum or other sample is determined using an immunoassay, such as an ELISA assay.

In some embodiments, the methods comprise comparing the level of SLC34A2 in situ in living muscle tissue, blood, plasma, serum or other sample to a level of SLC34A2 in one or more control samples.

In some embodiments, a difference in the level of SLC34A2 in the blood, plasma, serum or other sample from the subject compared with the level of SLC34A2 in one or more control blood, plasma, serum or other samples corresponds to SLC34A2 expression from muscle tissue in the subject.

In some embodiments, one or more control samples is from a subject that does not have FSHD. In some embodiments, one or more control samples is from a subject that has FSHD. In some embodiments, the subject is diagnosed as having FSHD.

In some embodiments, the level of SLC34A2 expression is monitored one or more times in the subject. Monitoring the expression of SLC34A2 over time can be advantageous to assess development of FSHD in the subject, assess the responsiveness to one or more therapies, and assay for therapeutic potential of one or more treatments. In some embodiments, SLC34A2 expression is assayed from 1-10 times. In some embodiments, the expression is assessed weekly, about twice a month, monthly, semi-annually, or annually.

In some embodiments, SLC34A2 expression can be detected using an antibody. As used herein, the term “antibody” encompasses antibodies and antibody fragments thereof, derived from any antibody-producing mammal (e.g., mouse, rat, rabbit, and primate including human), that specifically bind to, e.g., SLC34A2 polypeptides or portions thereof. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof.

The particular antibody that can be used is not particularly limiting provided it recognizes SLC34A2. In some embodiments, the antibody recognizes an extracellular epitope of SLC34A2. In some embodiments, the antibody is humanized. In some embodiments, the antibody is antibody MX-35. See, e.g., Yin et al., Monoclonal antibody MX35 detects the membrane transporter NaPi2b (SLC34A2) in human carcinomas. Cancer Immun. 2008 Feb. 6;8:3. PMID: 18251464; PMCID: PMC2935786, which is incorporated by reference in its entirety. Humanized MX-35 antibody is also known as Rebmab200. See Lopes dos Santos et al., PLoS One. 2013 Jul 31;8(7):e70332. doi: 10.1371/journal.pone.0070332. PMID: 23936189; PMCID: PMC3729455, which is incorporated by reference herein. In some embodiments, the antibody is Rebmab200 Antibody MX35 has been described to bind SLC34A2 present in epithelia and cancers. MX35 binds to intact cells and is FDA approved. Rebmab200 has been used to track metastasis and for ovarian cancer immunotherapy. Various SLC34A2 antibodies are available commercially. See, e.g., antibodies #80309 and #66445 from Cell Signaling Technology (Danvers, MA); Invitrogen (Waltham, MA) Cat. Nos.: PA5-58198, PA5-78333, PA5-143910, PA5-100689, PA5-50528, PA5-117157, and PA5-139878; Osenses (Keswick SA 5035, Australia) Cat. No.: OSS00417W-100UL and OS S 00416W-100UL ; Proteintech (Rosemont, IL) Cat. No.: 21295-1-AP.

In some embodiments, the agent that binds to SLC34A2 can be labeled to facilitate detection. In some embodiments, the label is a fluorescent label, an enzymatic label, or a radiolabel. The labeling can be direct or indirect, e.g., an antibody that binds to SLC34A2 can be recognized by a labeled antibody. In some embodiments, the labeled agent is detectable by PET or SPECT imaging. In some embodiments, the label is a fluorescent label.

In some embodiments, the SLC34A2 expression is quantitated. Methods for quantitating are known in the art and can include quantitative PCR assays. In some embodiments, the intensity of any label can be quantitated, e.g., a radiolabel or fluorescence label by imaging analysis.

In certain embodiments, a fluorescently-tagged MX35 antibody can be used to label myofibers in mice that express DUX4 transgenically, following viral transduction, or that have been xenografted to develop into mature human FSHD myofibers. Use of different fluorescent tags followed by in vivo imaging with IVIS instrumentation will allow for the monitoring and appearance of fibers expressing SLC34A2 as a biomarker for FSHD, and for following the progression of SLC34A2 expression in individual muscles and from muscle to muscle.

In some embodiments, the method comprises administering one or more treatments to the subject to treat a muscle disorder and following the effects of treatment by monitoring SLC34A2. In preferred embodiments, the condition is FSHD. The treatment can include administration of an effective amount of an agent or other therapy to the subject. In some embodiments, the treatment comprises one or more palliative treatments, such as therapy and/or agents which lead to increased muscle mass. In some embodiments, the agent to treat FSHD is a test agent, wherein the method is for identifying or screening for therapeutic agents capable of treating FSHD.

As used herein, the term “effective amount” is an amount of an agent that alleviates, totally or partially, the pathophysiological effects of the muscle disorder, such as FSHD. The amount will depend on, for example, the subject size, gender, magnitude of the associated condition or injury, and the like. For a given subject in need thereof, a therapeutically effective amount can be determined by those of ordinary skill in the art by methods known to those of ordinary skill in the art. The terms “effective amount” and “therapeutically effective amount” are used interchangeably.

As used herein, the term “treat” and all its forms and tenses refer to both therapeutic treatment and prophylactic or preventative treatment.

In some embodiments, the agent to treat the muscle disorder, such as FSHD, is not limiting. In some embodiments, the agent is a p38 kinase inhibitor, an agent that can inactivates DUX4 expression (e.g., nucleic acid nuclease), small molecules that activate methylation of the D4Z4 region, DUX4 surrogate targets and oligonucleotides, some linked to antibodies that bind to surface receptors on muscle fibers. In some embodiments, the agent to treat FSHD comprises a nucleic acid. The mode of delivering nucleic acids is not limiting and can include, e.g. viral vectors, such as AAV-based viral vectors, nanoparticles (e.g., lipid nanoparticles), extracellular vesicles, exosomes, or other vectors.

In some embodiments, the agent to treat FSHD suppresses the activity of DUX4. In some embodiments, the agent to treat FSHD reduces the expression of DUX4. In some embodiments, the agent comprises nucleic acids that can silence gene expression, such as RNA interference-based products, CRISPR-based products, U7 snRNAs and the like, for inhibiting or downregulating the expression of double homeobox 4 (DUX4). See, e.g., WO 2022/115745 A1, which is incorporated by reference herein. The safety and efficacy of DUX4 silencing using RNAi-based gene therapy delivered by AAV vectors in pre-clinical studies has been shown previously (Wallace et al., Mol Ther Methods Olin Dev 8, 121-130 (2018)).

In some embodiments, the agent is an inhibitor of p38 kinase. In some embodiments, the agent is an inhibitor of p38β and/or p38β such as a selective inhibitor of p38α or a selective inhibitor of p38β. In some embodiments, the inhibitor of p38 modulates the expression of DUX4. The inhibitor of p38 may not inhibit the MK2 pathway. In other embodiments, the inhibitor of p38 does not inhibit either p38δ or p38γ. In some embodiments, the inhibitor of p38 may be selected from acumapimod, ARRY-371797, pexmetinib, AS1940477, BMS-582949, dilmapimod, dorimapimod, losmapimod, LY2228820, LY3007113, pamapimod, PH-797804, SB202190, SB203580, TAK-715, talmapimod, VX-702, and VX-745. In an exemplary embodiment, the inhibitor of p38 is losmapimod. In some embodiments, the agent to treat FSHD comprises a P38 kinase inhibitor. In some embodiments, the p38 kinase inhibitor is losmapimod, pamapimod or LY222820 (Ralimetinib). See, e.g., U.S. Patent Appl. Pub. No.: US 2020/0297696 A1, which is incorporated by reference herein.

In some embodiments, a labeled MX35 antibody can be used to analyze SLC34A2 expression and determine the efficacy of treating mice with different p38 kinase inhibitors such as losmapimod which reduces the DUX4 program.

In some embodiments, the agent is a BET inhibitor such as I-BET762, I-BET726, I-BET151, RVX-208, CPI-203, CPI-232, CPI-0610, (+) JQ1, OTX-015, GW-841819X, BET-BAY-022, SRX-2523, or ABBV-075. In other embodiments, the second therapy is a β-2 adrenergic receptor agonist such as bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol. In some embodiments, the agent comprises a combination of p38 kinase inhibitor, BET inhibitor and β-2 adrenergic receptor agonist such as when the BET inhibitor is I-BET762, I-BET726, I-BET151, RVX-208, CPI-203, CPI-232, CPI-0610, (+) JQ1, OTX-015, GW-841819X, BET-BAY-022, SRX-2523, or ABBV-075 and the β-2 adrenergic receptor agonist is bitolterol, fenoterol, isoprenaline, levosalbutamol, orciprenaline, pirbuterol, procaterol, ritodrine, salbutamol, bambuterol, formoterol, arformoterol, clenbuterol, salmeterol, abediterol, indacaterol, or olodaterol.

In some embodiments, the therapy is a therapy to increase muscle mass, physical therapy, or occupational therapy. In some embodiments, the therapy is a therapy which improves the quality of life. In some embodiments, the therapy is scapular fusion or scapular bracing. In other embodiments, the therapy is an anti-inflammatory compound such as an NSAID or a glucocorticoid receptor modulator (glucocorticoid).

The route of administration, formulation, dosing and number of administrations is not limiting. The actual dosage amount of a therapeutic agent administered to a subject or patient can be determined by physical and physiological factors such as body weight, severity of condition, the type of disease being treated, previous or concurrent therapeutic and/or prophylactic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

The treatments may include various “unit doses.” Unit dose is defined as containing a predetermined quantity of the therapeutic composition calculated to produce the desired responses in association with its administration, e.g., the appropriate route and treatment regimen.

In some embodiments, the expression of SLC34A2 in living muscle tissue or blood, serum, plasma or other sample is detected following administration of the agent or therapy to treat the muscle disorder. In some embodiments, expression of SLC34A2 is detected one or more times following treatment. In some embodiments, expression of SLC34A2 is detected 2, 3, 4, 5, 6, 7, 8, 9, 10 or more times following treatment.

In some embodiments, the SLC34A2 expression in living muscle tissue, blood, serum, plasma or other sample following administration of the agent to treat FSHD is compared with SLC34A2 expression in living muscle tissue, blood, serum, plasma or other sample prior to administration of the agent to treat FSHD. In some embodiments, the SLC34A2 expression is compared to a control. In some embodiments, the control is from a healthy subject. As used herein, the term “human subject” refers to an individual who is known not to suffer from FSHD. In some embodiments, the control is from a subject having FSHD.

All publications herein are incorporated by reference to the same extent as if each individual publication or patent application was specifically and individually indicated to be incorporated by reference.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLES

### Example 1. SLC34A2 as a Biomarker for Facioscapulohumeral Muscular Dystrophy

The present example shows that SLC34A2 is a useful biomarker for FSHD. The data show that: (a) SLC34A2 protein is present in the myofibers of FSHD biopsies and FSHD xenografts at significantly higher levels than in controls; (b) SLC34A2 protein is detectable in sera of FSHD patients and in mice carrying FSHD xenografts at higher levels than in controls. The aim of this example is to determine if SLC34A2 protein is a reliable biomarker for the severity of FSHD and for the reduction of the DUX4 program by potential therapies.

There are four specific parts: (i) develop quantitative assays for SLC34A2 in myotubes; (ii) use the most promising assays from part (i) to determine the amounts of SLC34A2 in xenograft tissue, in serum from mice carrying xenografts, and in serum from individuals with FSHD; (iii) learn if SLC34A2 levels in the serum of engrafted mice diminish as the DUX4 program is suppressed by small molecular drugs, oligonucleotides and reagents introduced via adeno-associated virus; (iv) compare the levels of SLC34A2 in serum individuals with FSHD with their genetic status and severity of disease.

We screened nearly 20 different antibodies to proteins thought to be upregulated by the DUX4 program to determine if they could detected at elevated levels in xenografts of FSHD muscle compared to control grafts. The only one that did was to SLC34A2. SLC34A2 is a Na, phosphate cotransporter that is primarily expressed in epithelial tissues, with low to nondetectable levels in muscle. As epithelial tissues typically slough dead cells into their lumens, not into the blood, there is a significant possibility that much of the SLC34A2 that can be detected in FSHD serum arises from dead or dying muscle fibers.

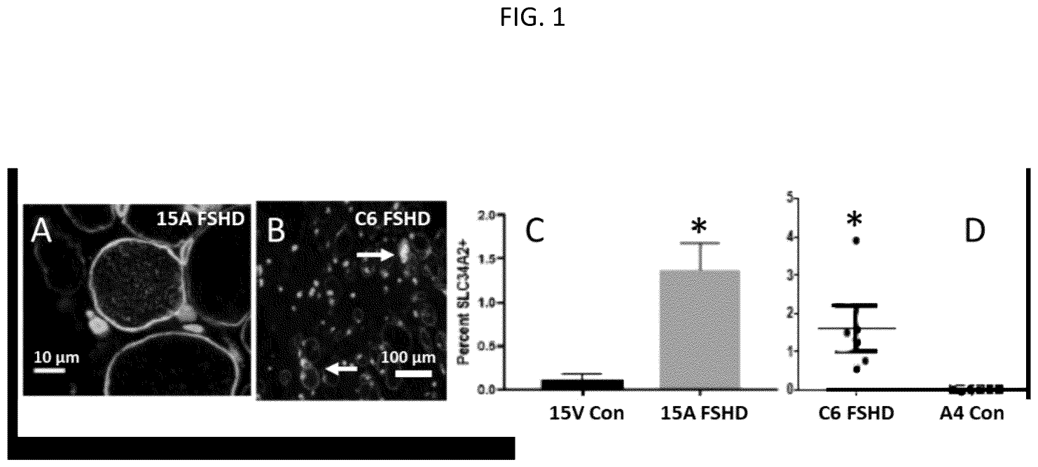

SLC34A2 protein detected in mature FSHD muscle fibers: FIG. 1 shows that frozen sections of xenografts prepared from FSHD cells (the 15A cell line developed by the Boston Wellstone group; 15V is a control from a healthy sibling) showed a significant increase in the number of myofibers that labeled for SLC34A2 with a commercial rabbit monoclonal antibody. FIG. 1 shows additional results obtained with a second pair of cell lines, C6 FSHD and A4 controls, provided the Mouly and van der Maarel laboratories, that show similar results. Thus, the SLC34A2 protein, or fragments thereof (can be readily detectable in FSHD myofibers at significantly higher levels than in controls. These increases are paralleled by increases in mRNA levels.

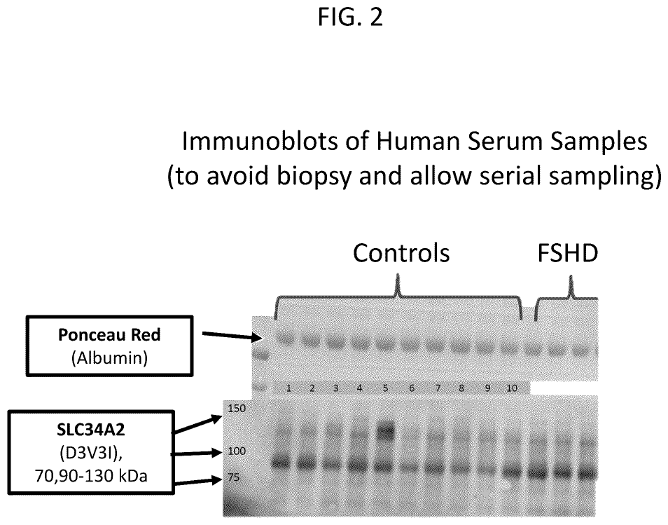

SLC34A2 protein detected in immunoblots: Our data indicate that we can detect greater numbers of myofibers labeled for SLC34A2 protein in biopsies of FSHD muscle than in controls. We used the same antibodies as in Fig.1 in immunoblots of COST cells transfected to express SLC34A2 (lane M), 15A myotubes in culture (lane 1), xenografts formed by the 15A cells (lane 2), and serum from mice carrying either 15A (FSHD: lane 3) or 15V (Con: lane 4) grafts. The results (FIG. 2) show that the intact SLC34A2 protein runs with an apparent molecular mass of ˜75-80 kDa (as expected

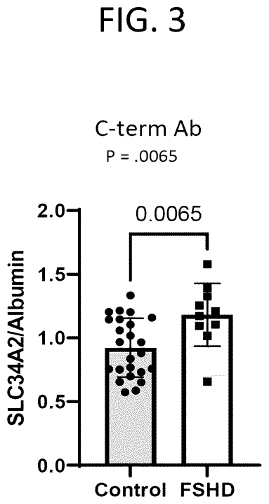

SLC34A2 detected in serum of FSHD patients: We obtained FSHD sera from Dr. J. Statland (KUMC) and purchased control serum. We subjected all samples to SOS-PAGE and immuno-blotting, as in FIG. 2, and quantitated the intensities of the SLC34A2 bands relative to a loading control, human serum albumin, visualized with Ponceau Red. Results, shown in FIG. 3, show that several FSHD sera tend to have higher relative amounts of SLC34A2 than the controls.

We quantitated the results from multiple FSHD and control samples. The results, shown in FIG. 3, indicate that serum from FSHD patients has a significantly higher amount of SLC34A2 than serum from healthy controls.

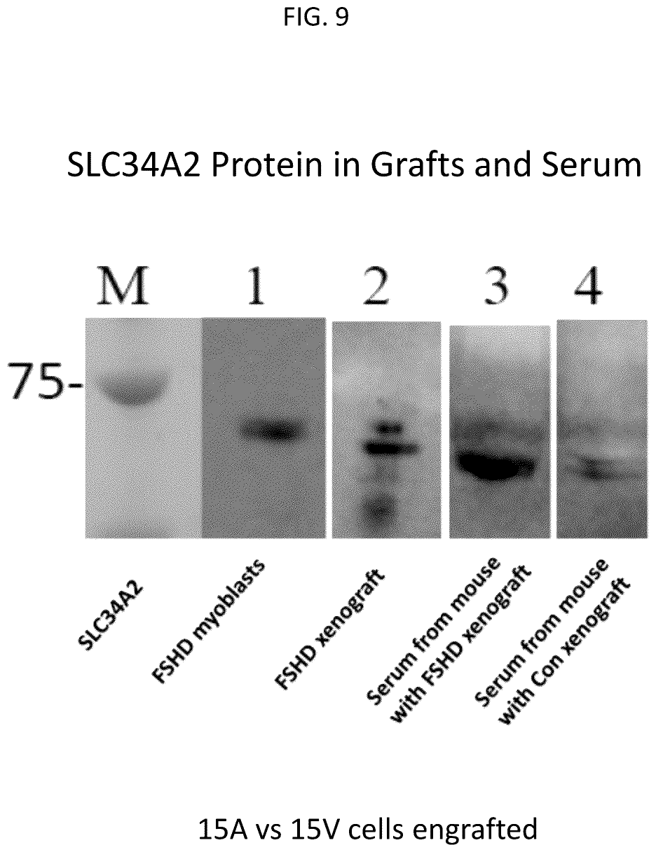

We also studied grafts and serum from mice engrafted to form muscle tissue from either FSHD or control human myoblasts. The results FIG. 9 show that SLC34A2 protein, or large fragments thereof, can be detected in FSHD tissue, in serum of mice carrying FSHD tissue, and in serum from mice carrying FSHD xenografts, at higher levels than in serum from mice carrying control xenografts

These observations establish the experimental justification, designed to measure SLC34A2 levels quantitatively and then assess its usefulness as a biomarker for FSHD.

### Experimental Approach

(i): Develop quantitative assays for SLC34A2 in myotubes. We will use a multipronged approach. Commercial antibodies are available to the C-terminal and N-terminal regions of the human SLC34A2 protein. We have found that both react well with hSLC34A2 in immunoblots and react specifically with their respective antigenic regions, prepared fusion proteins.

We currently have 2 immortalized FSHD cell lines, 15A and C6, and 2 immortalized control cell lines, 15V and A4 to use in these studies. 15A and 15V were obtained from siblings. C6 and A4 were from a patient for FSHD, providing an even closer genetic match. We will use all 4 lines in these experiments, Each will be grown to confluency in the medium we use prior to xenografting, and allowed to fuse to form myotubes.

Samples will be collected by scraping, pelleted, washed briefly in buffer to reduce contamination by the growth medium, and solubilized in either SOS-PAGE sample buffer at 37° C. (optimal temperature, determined in our preliminary studies), for WES Protein Simple, or in RIPA buffer, in preparation for MS/MS and ELISA assays.

Our first and major approach will use WES Protein Simple. Antibody reactions with fusion proteins will be assayed at different protein and antibody concentrations to create standard curves. We will then assay extracts of FSHD and control myotubes to determine the amounts in each (in μg SLC34A2/mg total protein).

An alternative approach, for comparison with WES, will use MS/MS (in collaboration with Dr. M Kane in our Proteomics Core). COST cells will be transfected to expressed SLC34A2. Two days later, the protein will be partially purified by immunoprecipitation with the commercial antibodies we've used to date. IPs will be solubilized in SOS-PAGE sample buffer at 37° C. and separated by electrophoresis. The SLC34A2 protein (75 kDa) should separate well from the immunoglobulin subunits and be readily identifiable with Coomassie Blue staining. The band will then be eluted and subjected to MS/MS, following procedures that are standard in our Proteomics Core. This should identify the peptides best suited for analysis of SLC34A2 in complex mixtures. These peptides will then be quantitated in protein extracts of FSHD and control myotubes.

A second alternative, also for comparison with WES, will utilize ELISA methods, employing a fusion protein containing both the N- and C-terminal epitopes flanking an MBP moiety to establish a standard curve. In particular, one antibody (e.g., to the N-terminus), will be captured on an ELISA plate. The fusion protein will be incubated with the coated plates. The plates will then be washed and incubated with the second antibody (to the C-terminus), tagged with biotin. Visualization will be with a colorimetric assay using an enzyme-coupled streptavidin. Once the standard curve is established, this approach will be applied to myotube extracts.

Ultimately, this approach could be used in Meso Scale Discovery assays, with multiplexing for control proteins that should not change in FSHD samples.

We expect that all 3 methods will give results that are quantitatively identical, or nearly so. We will select the two best methods (that give the least variability and that are quantitatively in agreement) for use in our later aims. Given our preliminary immunoblotting results, we expect WES to be one of these two methods.

(ii) Determine the amounts of SLC34A2 in xenograft tissue, in serum from mice carrying xenografts, and in biopsies and serum from individuals with FSHD. The methods developed in part (i) should allow us to quantitate the absolute amounts of SLC34A2 in different biological samples, as μg SLC342/mg protein. We will select the two assays that are most accurate for use in this aim.

We will use the cell lines described in part (i) to generate FSHD and control xenografts, using our established methods. A month after initial engraftment we will euthanize the mice, collect the grafts from the two TA compartments and collect serum. Samples will be processed as described in Aim (i) and subjected to quantitative assays. Biopsy and serum samples from individuals with FSHD and from healthy individuals or individuals with other neuromuscular diseases, obtained from Drs. Statland and Tawil, will be studied similarly.

To optimize our use of biopsy materials, which are limited, we will collect samples by cryosectioning.

Based on our experiments to date, we predict that these experiments will reveal increases of SLC34A2 of several fold or more in the FSHD samples, compared to controls and that these increases will be both quantitative and reproducible across methods.

(iii): Determine if SLC34A2 levels in the serum of engrafted mice diminish as the DUX4 program is suppressed by small molecular drugs, oligonucleotides and reagents introduced via adeno-associated virus. Suppression of the DUX4 program should reduce the expression of DUX4's downstream target genes, including SLC34A2. If we can reliably measure SLC34A2 in the serum of mice carrying xenografts, as described in Aim (i), we should be able to detect decreases in SLC34A2 as a function of time after suppression of DUX4 expression.

There are now several experimental approaches to reducing the DUX4 program, including the use of p38a kinase inhibitors, inactivate CRISPR as a silencer, delivered by AAV, small molecules that activate methylation of the D4Z4 region, DUX4 surrogate targets, delivered by AAV, and oligonucleotides, some linked to antibodies that bind to surface receptors on muscle fibers.

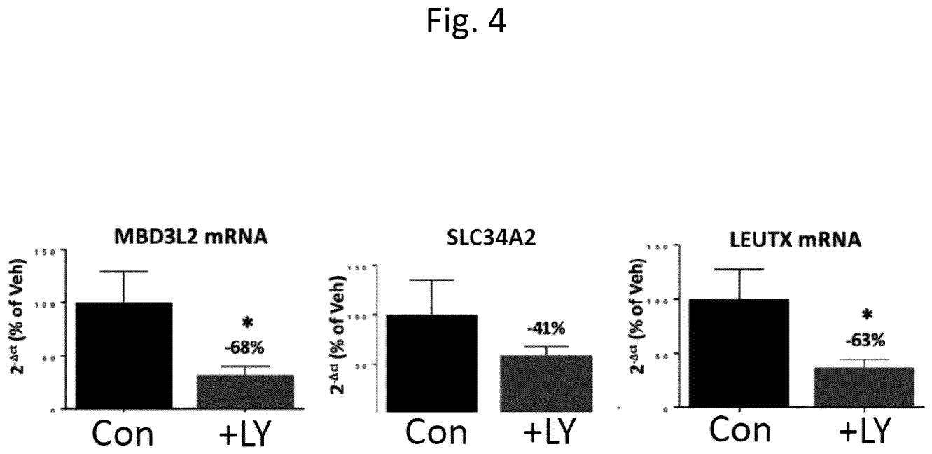

Xenografts are treated starting at 5 weeks after initial engraftment. Dosage is 10 mg/kg BID, injected IP. The qPCR results (FIG. 4) showed that MBD3L2 and LEUTX are reduce in expression by 60-70% (p<0.05) and that SLC34A2 is reduced in expression by 41%.

The experimental design of this part is similar to that for the LY compound, with small modifications:
Prepare grafts of 15A or C6 cells in 16 mice, using both hindlimbs; include 4 weeks of intermittent neuromuscular electrical stimulation, to yield the largest grafts possible given our current methods;
Treat 8 mice with small molecule at published doses, injected IP; or antibody-oligonucleotide conjugates at doses recommended by the biopharma company or laboratory involved, injected IV; or AAV at ˜2×10¹³ vg/mouse, injected retroorbitally. Controls (n=8) treated similarly, but with vehicle, inactive antibody- oligonucleotide conjugates, or AAV encoding sequences independent of DUX4.
Euthanize mice 1, 5 and 9 weeks after treatment with drug, antibody or virus; collect serum and tissue in xenografted TA compartment. For those mice carried for longer than 1 wk posttreatment, collect ˜150 μI blood from facial vein at 1 or 1 and 5 weeks, to track serum levels of SLC34A2 over time;
Analyze SLC34A2 in serum as in (i). Determine number of SLC34A2-positive fibers in sections of grafts (as % total human fibers). Assay DUX4 downstream products (e.g., SLC34A2, LEUTX, ZSCAN4, MBD3L2, TRIM43) by qPCR of graft extracts and, if sample size permits, SLC34A2 protein in graft extracts.
Compare effects of treatments on SLC34A2 levels in serum with effects on % myofibers labeled with anti-SLC34A2 antibodies by immunofluorescence and % reduction in mRNA levels of SLC34A2 and other DUX4 downstream targets.

(iv); Compare the levels of SLC34A2 in serum of individuals with FSHD with their genetic status and severity of disease. The severity of FSHD tends to increase as an inverse function of the size of the D4Z4 repeat region. If the results of our preceding aims establish that SLC34A2 is a valid biomarker for FSHD, then its levels in sera of individuals with FSHD should correlate with the repeat length and disease severity.

Quantitative values will be provided for: D4Z4 repeat length; functional strength (single time point), electrical impedance myography (EIM); MRI of 3 muscles (vastus, TA, gastrocnemius). Serum levels of SLC34A2 will be determined, as in part (ii), and should vary consistently with respect to each of these parameters. If serum SLC34A2 is indeed a reliable biomarker of FSHD, we should find a strong correlation (r value >0.5) in graphs of serum levels vs some or all of these parameters. For example, serum levels should increase as an inverse function of repeat length, and as a direct function of loss of muscle mass seen in MRI.

### Example 2. SLC34A2 Expression in Facioscapulohumeral Muscular Dystrophy

This example has specific parts: (i) develop quantitative assays for SLC34A2 in myotubes; (ii) use the most promising assays from part (i) to determine the amounts of SLC34A2 in xenograft tissue, in serum from mice carrying xenografts, and in serum from individuals with FSHD; (iii) learn if SLC34A2 levels in the serum of engrafted mice diminish as the DUX4 program is suppressed by small molecular drugs, oligonucleotides and reagents introduced via adeno-associated virus; (iv) compare the levels of SLC34A2 in serum individuals with FSHD with their genetic status and severity of disease.

(i): We have adapted and improved our procedures to assay for SLC34A2 mRNA and protein in myotubes of immortalized myoblasts derived from FSHD and healthy individuals. To establish the validity of our assays, we use losmapimod and other p38 kinase inhibitors. We have shown further with immunoblots that losmapimod as well as other p38 kinase inhibitors also suppress SLC34A2 protein levels in cultured FSHD myotubes −2-fold. P values are <0.05. These results are consistent with the idea that the SLC34A2 protein can be used as a biomarker for FSHD.

We can use a Abby Protein Simple instrument for more quantitative analysis of immunoblots and will use this in our studies of SLC34A2 in cells, serum and tissues.

(ii): Given the level of variability we observe in our xenografts, and the small numbers of FSHD myofibers in the grafts generated by some of the FSHD cell lines we have been studying, we thought it important to be able to monitor the diseased myofibers in living mice. Our approach is to inject mice with an infrared fluorescent version of a monoclonal antibody to SLC34A2, MX-35, that recognizes an extracellular epitope of the protein and that has been used clinically with patients with ovarian cancer. This approach should allow us to estimate the lifetime of the fibers once they become positive for SLC34A2, and, using different IR fluorophores, what the rate of appearance of newly positive fibers is in our grafts. It could also be used to test potential treatments for FSHD both in mice and in clinical trials with FSHD patients.

Our results show that MX35-tagged fibers can be detected in FSHD but not control grafts both in living mice (visualized by IVIS technology) and after collection of the grafts and frozen sectioning. Optimal dosage of the mAb is 5-10 μg/mouse. Labeling of frozen sections shows about the same relative number of positive fibers in the graft as we've reported before (1-2% for 15A xenografts; values for C6 xenografts are higher, at ˜5%). The half-life of the labeled fibers is about 1 day.

### Mass Spec Results

We transfected COS7 cells with plasmid expressing full length SLC34A2, allowed expression to proceed for 2 days, then collected the cells and provided the pellets to the laboratory for analysis. Controls were transfected with empty plasmid. The laboratory identified four SLC34A2 peptides that stand out as distinct and easy to quantitate in this context:

| | | |
|---|---|---|
| Residues | 660-671 | APETFDNITISR |
| Residues | 586-597 | LQNWNFLPLWMR |
| Residues | 598-608 | SLKPWDAVVSK |
| Residues | 281-291 | VISQIAMNDEK |

### Example 3. Visualization of SLC34A2 Expression using with MX-35 Antibody

Here we propose to test the ability of fluorescently-tagged MX35 to label myofibers in mice that express DUX4 transgenically or that have been xenografted to develop mature: human FSHD myofibers. Use of different fluorescent tags followed by in vivo imaging with IVIS instrumentation will allow us to monitor the appearance of fibers expressing SLC34A2, and then to follow the progression of SLC34A2's (and thus DUX4′s) expression in individual muscles and from muscle to muscle. We will also determine: if MX35 labeling is reduced by treating engrafted. mice treated a p38 kinase inhibitor, losmapimod, which reduces the DUX4 program in vitro and. is now in clinical trial. Materials and Methods

Mice are from JAX. B6(Cg)-Gt(ROSA)26Sortm1.1 (DUX4*)Plj/J (FLExDUX4-ACTA 1-MCM, or FLExDUX4) expresses low levels of DUX4 but expresses higher levels with doxycycline treatment. NRG mice will be engrafted. All procedures are approved by the IACUC, U of MD, Baltimore.

Antibodies: MX35 is a mouse mAb from the Sloan-Kettering Research Institute. Biotinylation and conjugation of MX35 or its Fab fragments, to Alexa fluorophores from InVitrogen/ThermoFisher, will follow manufacturer's instructions. Rabbit mAbs to the N- and C-terminal regions of SLC34A2 (Cell Signaling) are used at 1:100 dilutions. Fluorescent secondary antibodies and streptavidin are available commercially.

Injection and Imaging: Mice will be injected IV with fluorescent mAb or Fab fragments either through the tail vein or retroorbitally, with 150 μg of the biotinylated antibodyfluorophore conjugate in 150 μgsaline. Imaging in a Xenogen IVIS Spectrum Optical In Vivo imaging System (Perkin Elmer), will begin 2 hr later, repeated daily for 3 days, and at longer intervals thereafter. Repeated injections will also be done at regular intervals, to be determined, based on the levels of the antibody-conjugate remaining in circulation.

Immunofluorescence: Mice will be euthanized and blood samples taken. Muscles will be collected and either snap frozen or processed for PCR or immunoblotting. Frozen muscles will be-sectioned on a cryostat and labeled for biotinylated MX35 with fluorescent streptavidin or antibodies to biotin. Double labeling with rabbit anti-SLC34A2 antibodies will be performed to confirm the identity of positive fibers. Additional labeling with human-specific antibodies (e.g., to β-spectrin, lamin followed by isoform-specific secondary antibodies will be used to confirm the identify of fibers of human origin in the xenografts, as: necessary.

PCR and Immunoblotting will follow procedures well established in our laboratory, using: muscle tissue and serum from treated and control mice.

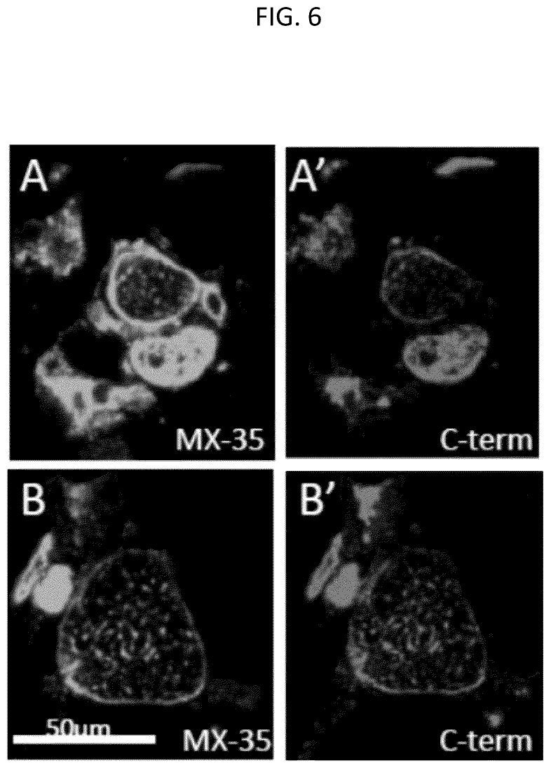

Results: We used immortalized C6 myoblasts (14) to generate xenografts in the Tibialis anterior muscles of NRG mice. Cross sections of the graft show similar immunofluorescent labeling by MX35 and a rabbit Ab to the C-terminus of SLC34A2 (FIG. 6). All labeled fibers also label with antibody to human β-spectrin (not shown), Control grafts (generated with A4 cells: 14) give no labeling (not shown), Thus, MX35 recognizes SLC34A2 in mature FSHD skeletal muscle. Importantly, it recognizes SLC34A2 in necrotic fibers (with intense cytoplasmic labeling) as well as in presumably pre-necrotic fibers (fibers with spots and thin lines in the myoplasm, as well as labeling at the sarcolemma) that still retain the reticular pattern of labeling typical of transverse tubules.

Experimental Plan and Anticipated Results: We will first biotinylate MX35 and then conjugate it to Alexa-Fluors 488, 554, 647 and/or infrared fluorophores and inject them into FLExDUX4 mice, IVIS imaging should reveal a significant signal in many muscles of the mouse that is significantly enhanced after dosing the mice with doxycycline. All 3 conjugates should give similar labeling, If they are inactive, however, it may be because they do not readily access the narrow lumenal spaces of the transverse tubules. (Transverse tubules in rat muscle were recently reported to have a mean inner diameter of ˜86 nm but tubules with diameters of ≤40 nm, also-present, were relatively impermeant to small proteins added to the solution. We expect the Fab-fragments prepared from the labeled MX35, with a Stokes radius of only 2.9 nm (less than half that of IgG), to penetrate these spaces even if intact IgG does not, yielding more intense signals.

The use of FLExDUX4 mice (or FLExDUX4 mice transduced with AAV to express SLC34A2 in skeletal muscle) for these studies will allow us to calibrate the extent of labeling. detected by IVIS with that detected by immunofluorescence labeling after collection of muscle and cryosectioning. Higher expression, driven by doxycycline, should be accompanied by parallel increases in labeling in situ by MX35 and in sections by the rabbit Ab to SLC34A2. These results would establish the validity of our methods. If our findings differ, our method is unlikely to prove useful either in clinical or pre-clinical settings.

However, if our method is validated by those studies, we will follow the fate of injected fluorescent MX35 over time to learn the kinetics of fiber death and dispersal. Sequential injections of mAbs with different fluorophores should further reveal if fibers with newly exposed SLC34A2 appear as the previously labeled fibers disappear. These studies in FLExDUX4 mice should also allow us to determine if different muscles are affected at different times after maturity, and if they degenerate at distinct rates in these mice, We will confirm these observations by immunofluorescent labeling, as time permits.

Once we have worked out our experimental paradigms with the FLExDUX4 mice, we will apply them to real FSHD tissue, grown in NRG mice according to our xenografting methods.

Administration of losmapimod (introduced by oral gavage at 12 mg/kg in 1 volume DMSO/9 volumes 0,5% methylcellulose, BID), which reduces the expression of SLC34A2 (10,11), should reduce the number and/or intensity of fibers in engrafted mice that label with MX35.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method of detecting SLC34A2 expression in living muscle tissue in a subject comprising
i) administering to the subject an effective amount of an agent that binds to SLC34A2 in the muscle tissue; and
ii) detecting the agent in the muscle tissue in situ, thereby detecting SLC34A2 expression in living muscle tissue.

2.-5.(canceled)

6. The method of claim 1, wherein the SLC34A2 expression in living muscle tissue is monitored over time in the subject.

7. The method of claim 1, wherein the SLC34A2 expression in living muscle tissue is quantitated.

8. The method of claim 1, wherein the subject is a human.

9. The method of claim 8, wherein the subject is diagnosed as having FSHD.

10. The method of claim 1, wherein the subject is administered an effective amount of an agent to treat FSHD.

11.-19. (canceled)

20. The method of claim 1, wherein the agent that binds to SLC34A2 comprises an antibody,

21-24 (canceled)

25. The method of claim 1, wherein the agent comprises a humanized form of the antibody MX-35 (Rebmab200).

26. A method of detecting SLC34A2 expression from muscle tissue in blood, plasma or serum from a subject comprising
i) providing a blood, plasma or serum sample from the subject; and
ii) conducting an assay to detect a level of SLC34A2 in the blood, plasma or serum sample.

27. The method of claim 26, wherein the assay comprises contacting the blood, plasma or serum sample with an agent that binds to SLC34A2, and detecting the binding.

28. (canceled)

29. (canceled)

30. The method of claim 26, wherein the method comprises comparing the level of SLC34A2 in the blood or serum sample to a level of SLC34A2 in one or more control blood or serum samples, wherein a difference in the level of SLC34A2 in the blood, plasma or serum sample from the subject compared with the level of SLC34A2 in one or more control blood, plasma or serum samples corresponds to SLC34A2 expression from muscle tissue in the subject.

31.-40. (canceled)

41. The method of claim 26, wherein the subject is diagnosed as having FSHD.

42. The method of claim 41, wherein the subject is administered an effective amount of an agent to treat FSHD.

43.-56. (canceled)

57. The method of claim 27, wherein the agent comprises a humanized form of the antibody MX-35 (Rebmab200).

58. A method of detecting SLC34A2 expression in muscle tissue in a sample from a subject comprising
i) providing the sample from the subject; and
ii) conducting an assay to detect an expression level of SLC34A2 in the sample.

59. The method of claim 58, wherein the assay comprises contacting the sample with an agent that binds to SLC34A2, and detecting the binding.

60.-63. (canceled)

64. The method of claim 58, wherein the method comprises comparing the level of SLC34A2 in the sample to an expression level of SLC34A2 in one or more control samples, wherein a difference in the level of SLC34A2 in the sample from the subject compared with the level of SLC34A2 in one or more control samples corresponds to SLC34A2 expression from muscle tissue in the subject.

65.-74. (canceled)

75. The method of claim 58, wherein the subject is diagnosed as having FSHD.

76. The method of claim 75, wherein the subject is administered an effective amount of an agent to treat FSHD.

77.-90. (canceled)

91. The method of claim 59, wherein the agent comprises a humanized form of the antibody MX-35 (Rebmab200).
